(19) United States
(12) Patent Application Publication
(10) Pub. No.: US 2024/0108401 A1
(43) Pub. Date: Apr. 4, 2024

(54) MULTI-FUNCTION ERGONOMIC HANDHELD SURGICAL INSTRUMENT AND SYSTEM

(71) Applicant: Covidien LP, Mansfield, MA (US)

(72) Inventors: Tong Shen, Shanghai (CN); Jun Zhu, Shanghai (CN); Guangcheng Luo, Shanghai (CN); Shoou Sheng Row, Shanghai (CN); Shunhong Xu, Shanghai (CN); Herbert Zhang, Shanghai (CN); Shenghua Yang, Shanghai (CN)

(21) Appl. No.: 18/274,756

(22) PCT Filed: Jan. 29, 2021
(86) PCT No.: PCT/CN2021/074406

§ 371 Date: Jul. 28, 2023

(51) Int. Cl. A61B 18/14; A61B 18/12

(52) U.S. Cl. CPC A61B 18/1482; A61B 18/1206; A61B 2017/00424

(57) ABSTRACT

A surgical instrument (10) includes a housing (20) defining a longitudinal axis and a first assembly (200) and a second assembly (300) operably coupled to the housing (20). The first assembly (200) includes a first function button (200b) pivotably coupled to the housing (20) about a first pivot (200p) and a first circuit (210) supported by the housing (20) and aligned with the first pivot (200p). The first function button (200b) includes a first arm (201) on a first side of the first pivot (200p) and a second arm (202) on a second side of the first pivot (200p). The first circuit (210) is aligned with the cutting picot between the first arm (201) and the second arm (202) and includes a first actuator (211) on the first side of the first pivot (200p) and a second actuator (212) on the second side of the first pivot (200p). The second assembly (300) includes a second function button (301) pivotably coupled to the housing (20) about a second pivot (300p) and a second circuit (310) supported by the housing (20) beneath the second function button (301).

### FIELD

The present disclosure relates generally to surgical instruments and, more specifically, to handheld surgical instruments for laparoscopic electrosurgery having a symmetrical button layout design for single-handed use.

### BACKGROUND

Laparoscopic surgery, a typical form of minimally invasive surgery (MIS), is sharing increasing surgery proportion due to its significant advantages to patients, which include smaller incisions, reduced pain, reduced hemorrhaging, and shorter recovery time.

Electrosurgical monopolar devices and suction/irrigator devices are the two most frequently used devices in laparoscopic surgery. A combination device that provides both electrosurgery and suction/irrigation functionality eliminates the need for creating multiple trocar holes and reduces the procedure time as multiple devices do not need to be exchanged through a single trocar hole.

Currently there exists two main types of electrosurgical suction and irrigation instruments, namely, a pencil style device and a pistol style device. The pencil style device, which is a design derived from the traditional monopolar device, remains popular with loyal users that shifted from open surgery to laparoscopic surgery. In the pencil style devices, the function buttons (e.g., cutting, coagulation, irrigation, suction, etc.) on the pencil handpiece commonly lay along the electrode axis from the proximal end of the device to the distal end of the device. The pistol style device remains popular among users that have adapted the more suitable and stable pistol grip design. In the pistol style device, the layout of the function buttons varies between different products, but are typically designed for right-handed users and/or require two hands to operate.

### SUMMARY

In accordance with aspects of the present disclosure, a surgical instrument for laparoscopic electrosurgery is provided. The surgical instrument has a symmetrical button layout design for single-handed use by both right-handed and left-handed users and includes electrosurgical cutting and coagulation functionality as well as suction and irrigation functionality. The design of the disclosed surgical instrument limits or eliminates the need for hand relocation when shifting between electrosurgical control buttons (e.g., cutting and coagulation) and fluid control buttons (e.g., irrigation and suction). The design of the disclosed surgical instrument also serves to stabilize a user's grip during performance of delicate tissue treatment procedures and minimizes a user's hand fatigue during use.

In accordance with an aspect of the present disclosure, a surgical instrument includes a housing, a first assembly, and a second assembly operably coupled to the housing. The first assembly includes a first function button pivotably coupled to the housing about a first pivot and a first circuit supported by the housing and aligned with the first pivot. The first function button includes a first arm on a first side of the first pivot and a second arm on a second side of the first pivot. The first circuit is aligned with the first pivot between the first arm and the second arm and includes a first actuator on the first side of the first pivot and a second actuator on the second side of the first pivot. The second assembly includes a second function button pivotably coupled to the housing about a second pivot and a second circuit supported by the housing beneath the second function button.

In an aspect, the first pivot defines a longitudinal axis that is parallel to a longitudinal axis of the housing such that the first function button is pivotable about the longitudinal axis of the housing and the second pivot defines a longitudinal axis that is perpendicular to the longitudinal axis of the housing.

In an aspect, each of the first actuator and the second actuator of the first circuit is configured to control delivery of electrosurgical cutting energy.

In an aspect, the first pivot includes a longitudinally extending protrusion configured to fit within a corresponding recess defined by the housing.

In an aspect, the second pivot includes a longitudinally extending protrusion configured to fit within a corresponding recess defined by the housing.

In an aspect, the surgical instrument further includes a suction and irrigation assembly operably coupled to the housing. The suction and irrigation assembly includes a dual chamber poppet valve defining a longitudinal axis extending at an angle relative to the longitudinal axis of the housing, an irrigation button operably coupled to a first chamber of the dual chamber poppet valve and extending through an opening defined through a top surface of the housing, and a suction button operably coupled to a second chamber of the dual chamber poppet valve and extending through an opening defined through a top surface of the housing.

In an aspect, the irrigation button and the suction button are disposed abreast on the top surface of the housing.

In accordance with an aspect of the present disclosure, a system for electrosurgical laparoscopic surgery is provided. The system includes an electrosurgical generator configured to generate electrosurgical energy and a surgical instrument configured to operably couple to the electrosurgical generator. The surgical instrument includes a housing, a first assembly, and a second assembly operably coupled to the housing. The first assembly includes a first function button pivotably coupled to the housing about a first pivot and a first circuit supported by the housing and aligned with the first pivot. The first function button includes a first arm on a first side of the first pivot and a second arm on a second side of the first pivot. The first circuit is aligned with the first pivot between the first arm and the second arm and includes a first actuator on the first side of the first pivot and a second actuator on the second side of the first pivot. The second assembly includes a second function button pivotably coupled to the housing about a second pivot and a second circuit supported by the housing beneath the second function button.

In an aspect, the first pivot defines a longitudinal axis that is parallel to a longitudinal axis of the housing such that the first function button is pivotable about the longitudinal axis of the housing and the second pivot defines a longitudinal axis that is perpendicular to the longitudinal axis of the housing.

In an aspect, each of the first actuator and the second actuator of the first circuit is configured to control delivery of electrosurgical cutting energy.

In an aspect, the first pivot includes a longitudinally extending protrusion configured to fit within a corresponding recess defined by the housing.

In an aspect, the second pivot includes a longitudinally extending protrusion configured to fit within a corresponding recess defined by the housing.

In an aspect, the system further includes a suction system configured to generate vacuum pressure and an irrigation system storing irrigation fluid. The surgical instrument is configured to operably couple to the suction system and the irrigation system and includes a suction and irrigation assembly operably coupled to the housing. The suction and irrigation assembly includes a dual chamber poppet valve defining a longitudinal axis extending at an angle relative to the longitudinal axis of the housing, an irrigation button operably coupled to a first chamber of the dual chamber poppet valve and extending through an opening defined through a top surface of the housing, and a suction button operably coupled to a second chamber of the dual chamber poppet valve and extending through an opening defined through a top surface of the housing.

In an aspect, the irrigation button and the suction button are disposed abreast on the top surface of the housing.

In another aspect of the present disclosure, a surgical instrument is provided. The surgical instrument includes a housing, a first assembly, a second assembly, and a suction and irrigation assembly operably coupled to the housing. The first assembly includes a first function button pivotably coupled to the housing about a first pivot and a first circuit. The first function button includes a first arm positioned on a first side of the housing and a second arm positioned on a second side of the housing. The first circuit includes a first actuator a second actuator activatable by actuation of either of the first arm or the second arm of the first function button. The second assembly includes a second function button pivotably coupled to the housing about a second pivot and a second circuit supported by the housing beneath the second function button. The suction and irrigation assembly includes a dual chamber poppet valve defining a longitudinal axis extending at an angle relative to the longitudinal axis of the housing, an irrigation button operably coupled to a first chamber of the dual chamber poppet valve and extending through an opening defined on a top surface of the housing, and a suction button operably coupled to a second chamber of the dual chamber poppet valve and extending through an opening defined on a top surface of the housing.

In an aspect, the first pivot defines a longitudinal axis that is parallel to a longitudinal axis of the housing and the second pivot defines a longitudinal axis that is perpendicular to the longitudinal axis of the housing.

In an aspect, each of the first actuator and the second actuator of the first circuit is configured to control delivery of electrosurgical cutting energy.

In an aspect, the first pivot includes a longitudinally extending protrusion configured to fit within a corresponding recess defined by the housing.

In an aspect, the second pivot includes a longitudinally extending protrusion configured to fit within a corresponding recess defined by the housing.

In an aspect, the irrigation button and the suction button are disposed abreast on a surface of the housing.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a plan view of an electrosurgical suction and irrigation system in accordance with the present disclosure;

FIG. 2 is a side perspective view of a surgical instrument of the electrosurgical suction and irrigation system of FIG. 1 with portions thereof removed to illustrate components of the surgical instrument;

FIG. 3 is a front view of a first assembly of the surgical instrument of FIG. 2;

FIG. 4 is a side view of a portion of the surgical instrument of FIG. 2 exposing a second assembly thereof;

FIG. 5 is a side view of a portion of the surgical instrument of FIG. 2 with portions cut-away exposing a suction and irrigation assembly thereof;

FIG. 6 is a side view of the surgical instrument of FIG. 2 grasped by a user's hand illustrating the user's index finger and middle finger moving a wheel of the surgical instrument distally;

FIG. 7 is a side view of the surgical instrument of FIG. 2 grasped by a user's hand illustrating the user's middle finger moving the wheel of the surgical instrument proximally; and

FIG. 8 is a side view of the surgical instrument of FIG. 2 grasped by a user's hand illustrating the user's index finger placed on a second function button of the surgical instrument.

### DETAILED DESCRIPTION

Particular embodiments of the present disclosure are described below with reference to the accompanying drawings. In the following description, well-known functions or constructions are not described in detail to avoid obscuring the present disclosure in unnecessary detail.

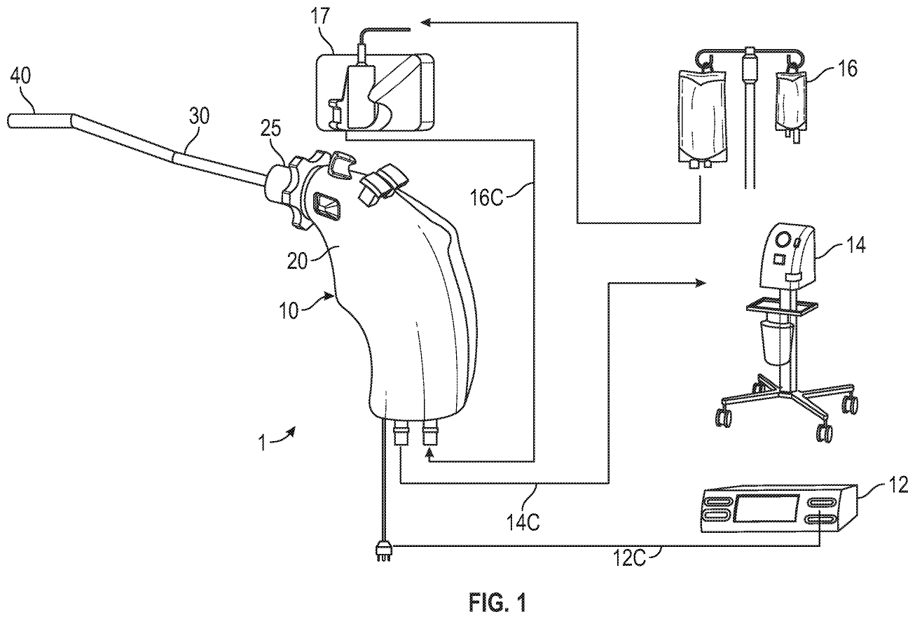

An electrosurgical laparoscopic system 1 according to the present disclosure is shown in FIG. 1. The electrosurgical system 1 includes a surgical instrument 10, an electrosurgical generator 12, a suction system 14, and an irrigation system 16 which may optionally include a pump 17. The surgical instrument 10 possesses electrosurgical cutting, electrosurgical coagulation, suction, and irrigation functionality and is configured to couple to the electrosurgical generator 12 via cable 12c, to the suction system 14 via tubing 14c, and to the irrigation system 16 via tubing 16c. Electrosurgical energy generated by the electrosurgical generator 12 is delivered to tissue by the surgical instrument 10 for cutting and coagulating the tissue. The suction system 14 creates a negative pressure suction force for removal of fluids and debris from a surgical site. The irrigation system 16 delivers fluid (e.g., liquid or gas) to the surgical site to clean portions of the surgical instrument 10 and/or to clear the surgical site of debris.

Surgical instrument 10 includes a pistol grip style housing 20, a wheel 25 rotatably coupled to the housing 20 and longitudinally movable relative to the housing 20, an elongated shaft 30 extending from the housing 20, and an electrode 40 disposed at a distal end of the elongated shaft 30. In an aspect, the elongated shaft 30 is fixed to the wheel 25 such that rotation of the wheel 25 causes corresponding rotation of the elongated shaft 30 and such that longitudinal movement of the wheel 25 in either the distal or proximal direction causes corresponding longitudinal movement of the elongated shaft 30. Elongated shaft 30 defines a lumen 31 through which a suction channel 32 in fluid communication with the suction system 14 extends to remove fluid and debris from the surgical site. Additionally, an irrigation channel 34 extends through the lumen 31 of the elongated shaft 30 to deliver irrigation fluid from the irrigation system 16 to the surgical site. The electrode 40 may be any suitably shaped electrocautery blade, for example, a cylindrical blade, a hook blade, a spatula blade, or any combinations thereof, and may be removably attached to the surgical instrument 20 so as to be interchangeable by a user.

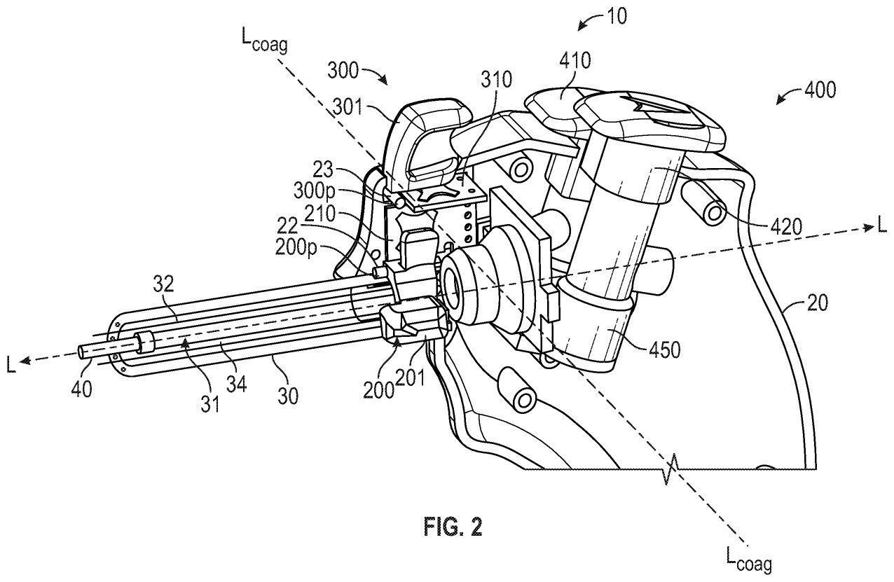

FIG. 2 illustrates some of the components of the surgical instrument 10 including a first assembly 200, a second assembly 300, and a suction and irrigation assembly 400. Each of the first assembly 200 and the second assembly 300 electrically couples the electrode 40 to the electrosurgical generator 12 (FIG. 1) and controls the delivery of electrosurgical energy from the electrosurgical generator 12 to the electrode 40. The suction and irrigation assembly 400 couples the suction channel 32 to the suction system 14 and the irrigation channel 34 to the irrigation system 16 and is used to control the amount and/or rate of fluid delivered to the surgical site and the amount and/or rate of fluid and debris removed from the surgical site.

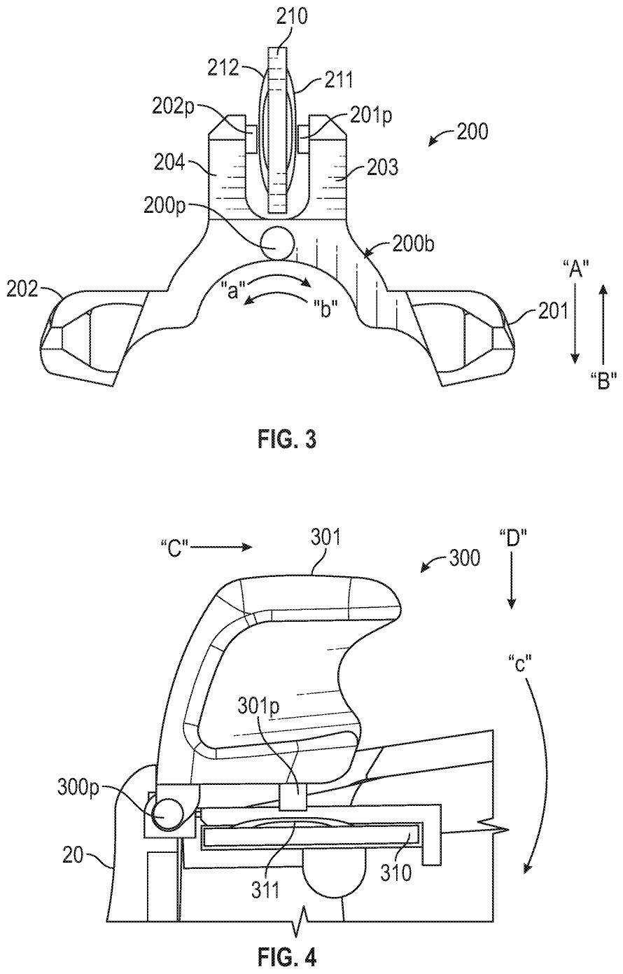

With reference to FIGS. 2 and 3, the first assembly 200 includes a first function button 200b, which is pivotably coupled to the housing 20 of the surgical instrument 10 via a first pivot 200p, and a first circuit 210 which is activatable by the first function button 200b to control delivery of electrosurgical energy (e.g., electrosurgical cutting energy) to the electrode 40. The first pivot 200p defines a longitudinal axis “Lcut” that is parallel to a longitudinal axis “L” defined by the housing 20, thereby enabling the first function button 200b to pivot about the longitudinal axis “L” of the housing 20. In an aspect, the first pivot 200p defines a longitudinally extending protrusion which is inserted into a corresponding recess 22 defined in the housing 20 to pivotably couple the first function button 200b to the housing 20.

First function button 200b includes a first arm 201 located on a first side of the first pivot 200p and a second arm 202 located on a second side of first pivot 200p. First arm 201 extends through an opening defined through one side of the housing 20 and second arm 202 extends through an opening defined through the opposite side of the housing 20 for manipulation of the first function button 200b on either side of the housing 20. That is, the first function button 200b may be manipulated by of a left-handed user's index finger to manipulate the first arm 201 on the first side of the housing 20 or by a right-handed user's index finger to the manipulate second arm 202 on the second side of the housing 20. Downward depression (in the direction of arrow “A”) or upward lifting (in the direction of arrow “B”) of either the first arm 201 or the second arm 202 by a user causes the first function button 200b to pivot about the longitudinal axis “Lcut” defined by the first pivot 200p (e.g., in the direction of arrows “a” or “b”).

The first circuit 210 is supported by the housing 20 and aligned with the first pivot 200p within a recess defined between a prong 203 of the first arm 201 and a prong 204 of the second arm 202. The first circuit 210 includes a first actuator 211 on one of its sides and a second actuator 212 on its other side and is positioned relative to the first function button 200b such that the first actuator 211 is disposed on the first side of the first pivot 200p and the second actuator 212 is disposed on the second side of the first pivot 200p. The first actuator 211 and the second actuator 212 may each include a metal dome, button, switch or other device or combination of devices that create a short circuit on the first circuit 210 when depressed or otherwise activated.

With the disclosed configuration and relative positioning of the components of the first assembly 200, movement of either of the first arm 201 or the second arm 202 of the first function button 200b in any direction, and from either side of the housing 20 of the surgical instrument 10, causes at least one of a protrusion 201p extending laterally from the first prong 203 or a protrusion 202p extending laterally from the second prong 204 to actuate at least one of the first actuator 211 or the second actuator 212 of the first circuit 210. For example, when a right-handed user is operating the surgical instrument 10, the user can easily control (e.g., initiate, cease, or modify) delivery of electrosurgical energy to the electrode 40 by depressing the second arm 202 downward (in the direction of arrow “A”) with the user's right index finger. Alternatively, when a left-handed user is operating the surgical instrument 10, the user can easily control (e.g., initiate, cease, or modify) delivery of electrosurgical energy to the electrode 40 by depressing the first arm 201 downward (in the direction of arrow “A”) with the user's left index finger.

With reference to FIGS. 2 and 4, the second assembly 300 includes a second function button 301 that is pivotably coupled to the housing 20 of the surgical instrument 10 via a second pivot 300p and a second circuit 310 that is activatable by the second function button 301 to control delivery of electrosurgical energy (e.g., electrosurgical coagulation energy) to the electrode 40. In an aspect, the second pivot 300p defines a longitudinally extending protrusion that is inserted into a corresponding recess 23 defined in the housing 20 to pivotably couple the second function button 301 to the housing 20. The second pivot 300p defines a longitudinal axis “Lcoag” that is perpendicular to the longitudinal axis “L” defined by the housing 20, thereby enabling the second function button 301 to pivot about the longitudinal axis “Lcoag” of the second pivot 300p when pulled back (or pressed down) by a user's index finger. Second function button 301 extends through an opening defined through a top side of the housing 20, toward a distal portion of the housing 20, for easy access and manipulation by a user's index finger without sacrificing the integrity of the user's grip on the other portions of the housing 20 and while minimizing hand fatigue caused by, for example, movement of the user's finger. This specific positioning of the second function button 301 also enables equal accessibility for manipulation of the second function button 301 by the index finger of both right-handed users and left-handed users, that is, by a right-handed user's right index finger and a left-handed user's left index finger. In particular, proximal movement of the second function button 301 (in the direction of arrow “C”) or downward movement of the second function button 301 (in the direction of arrow “D”) caused by a right-handed user's right index finger or by a left-handed user's left index finger serves to pivot the second function button 301 about the longitudinal axis “Lcoag” defined by the second pivot 200p (e.g., in the direction of arrow “c”).

The second circuit 310 is supported by the housing 20 of the surgical instrument 10 beneath the second function button 301 and includes an actuator 311 disposed on a top surface thereof facing a protrusion 301p that extends from a bottom surface of the second function button 301. The actuator 311 may include a metal dome, button, switch or other device or combination of devices which creates a short circuit on the second circuit 310 when depressed or otherwise activated.

With the disclosed configuration and relative positioning of the components of the second assembly 300, a variety of movements by the index finder of either a right-handed user or a left-handed user can be used to actuate the second function button 301. In particular, proximal movement (in the direction of arrow “C”) of the second function button 301 by either a right-handed user's right index finger when gripping the housing 20 of the surgical instrument 10 with the right hand or a left-handed user's left index finger when gripping the surgical instrument 20 with the left hand, serves to pivot the second function button 301 about the longitudinal axis “Lcoag” (in the direction of arrow “c”) to actuate the actuator 311 and control delivery of electrosurgical coagulation energy to the electrode 40. Additionally, with the disclosed configuration and relative positioning of the components of the second assembly 300, downward depression (in the direction of arrow “D”) of the second function button 301 by either a right-handed user's right index finger when gripping the surgical instrument 10 with the right hand or a left-handed user's left index finger when gripping the surgical instrument 20 with the left hand, also causes the second function button 301 to pivot about the longitudinal axis “Lcoag” (in the direction of arrow “c”) to actuate the actuator 311 and control delivery of electrosurgical coagulation energy to the electrode 40. Thus, a broad range of button movements caused by a user's index finger, that is any of proximal movement of the second function button 301 or downward depression of the second function button 301, actuates the actuator 311 of the second circuit 310 to control delivery of energy, for example, coagulation energy. Moreover, the central location of the second function button 301 on the top surface of the housing 20 provides both right-handed users and left-handed users the ability to actuate the second function button (either by downward depression or proximal movement).

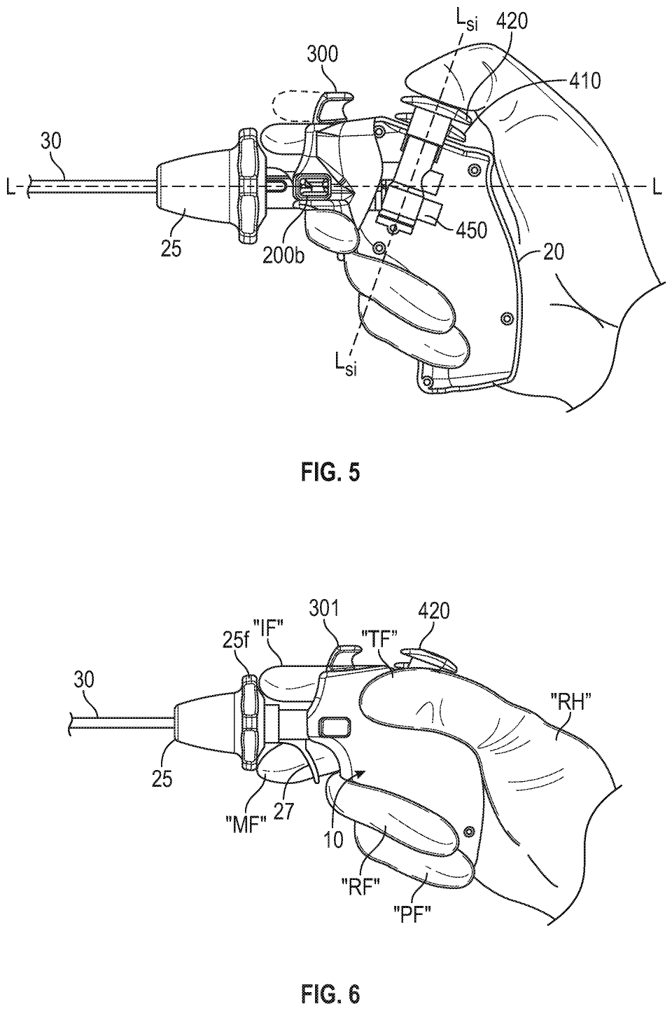

Referring to FIGS. 2 and 5, the surgical instrument 10 also includes a suction and irrigation assembly 400 for controlling suction from, and irrigation to, the surgical site or portions of the surgical instrument 10. The suction and irrigation assembly 400 includes a dual chamber poppet valve 450 which includes a first chamber 451 that is controllable by an irrigation button 410 for delivery of irrigation fluid to the surgical site from the irrigation system 16 (FIG. 1) and a second chamber 452 that is controllable by a suction button 420 for controlling suction of fluid and debris from the surgical site to the suction system 14 (FIG. 1).

The dual chamber poppet valve 450 defines a longitudinal axis “Lsi” extending at an angle “α” relative to the longitudinal axis “L” of the housing 20. The irrigation button 410 and the suction button 420 extend from the first chamber 451 and the second chamber 452, respectively, through one or more openings defined through a top surface of the housing 20 toward a proximal portion of the housing 20. The abreast layout of the irrigation button 410 and the suction button 420 at a proximal portion of the top surface of the housing 20 at the angle “α” relative to the longitudinal axis “L” of the housing 20 enables a user's thumb to press each of the irrigation button 410 and the suction button 420 without stretching and/or impacting the user's grip on other portions of the housing 20, relieves thumb fatigue when constantly pressing and switching between the irrigation button 410 and the suction button 420, and eliminates component force. Additionally, as the first function button 200b and the second function button 301 are positioned to be controlled by a user's index finger, and the irrigation button 410 and the suction button 420 are positioned to be controlled by the user's thumb, the user when griping the housing 20 can easily activate the electrosurgical treatment functions (e.g., cutting and coagulation) and fluid control features (e.g., suction and irrigation) of the surgical instrument 10 simultaneously. Further, the positioning of the first function button 200b and the second function button 301 relative to each other on the housing 20 enable the user to easily switch between operating the different electrosurgical treatment functions (e.g., cutting and coagulation) with ease and with minimal hand fatigue. Likewise, the positioning of the irrigation button 410 and the suction button 420 relative to each other on the housing 20 enables the user to easily switch between operating the different fluid control features (e.g., suction and irrigation) with ease and minimal hand fatigue.

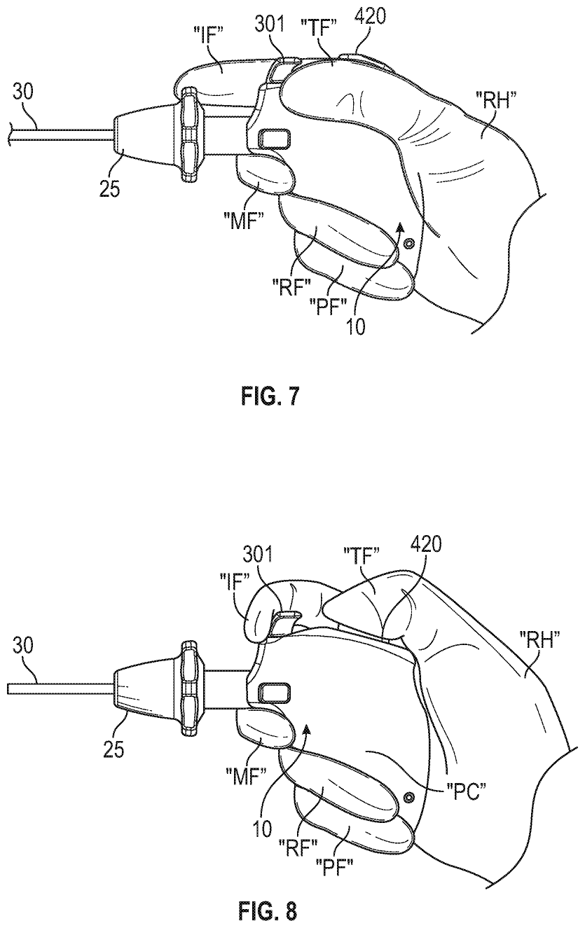

Continuing with reference to FIG. 5, and with additional reference to FIGS. 6-8, in an aspect, the design of the disclosed surgical instrument 10 designates convenient rest positions on the housing 20 for every finger of a user's hand, equally applicable to both right-handed users and left-handed users. The designated rest positions may be a shaped (e.g., molded) area defined in a particular area of the housing. Although FIGS. 6-8 illustrate a right-handed user's right hand “RH” gripping the surgical instrument 10, it is understood that all of the described movements and gripping or resting positions of the user's right hand “RH” are equally applicable and available to a left-handed user gripping the surgical instrument 10 with their left hand.

The index finger “IF” is assigned to trigger the second function button 301 and the first function button 200b and to deploy and rotate the wheel 25. The rest position of the index finger “IF” is on the front curve of the housing 20, which eliminates the accidental pulling back or downward depression of the second function button 301 or accidental depression of the first function button 200b. To initiate coagulation, the index finger “IF” moves up from the front curve of the housing 20, toward the front surface of the second function button 301, and pulls back or depresses down to activate the coagulation. To initiate cutting, the index finger “IF” moves down toward the second arm 202 (for a right-handed user) and presses down to activate the cutting.

To deploy the wheel 25, the index finger “IF”, together with the middle finger “MF”, push the flange 25f of the wheel and may continue further distally to distally advance the wheel 25 until a distal stop (not shown) is reached preventing further distal advancement of the wheel 25. The rest position of middle finger “MF” is on the front surface of a trigger 27, which is fixed to the wheel 25 and therefore moves distally and proximally with the wheel 25. The wheel 25 may be retreated proximally by pulling the trigger 27 back with the middle finger “MF”. The rest positions of the ring finger “RF” and pinkie finger “PF” are on the pistol curve “PC” to stabilize and hold the surgical instrument and are not compromised by movement of the thumb finger “TF”, the index finger “IF” or the middle finger “MF” during operation of the surgical instrument 10.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A surgical instrument comprising:
a housing;
a first assembly operably coupled to the housing and including:
a first function button pivotably coupled to the housing about a first pivot and including a first arm on a first side of the first pivot and a second arm on a second side of the first pivot; and
a first circuit supported by the housing and aligned with the first pivot between the first arm and the second arm, the first circuit including a first actuator on the first side of the first pivot and a second actuator on the second side of the first pivot; and
a second assembly operably coupled to the housing and including:
a second function button pivotably coupled to the housing about a second pivot; and
a second circuit supported by the housing beneath the second function button.

2. The surgical instrument of claim 1, wherein the first pivot defines a longitudinal axis that is parallel to a longitudinal axis of the housing such that the first function button is pivotable about the longitudinal axis of the housing and wherein the second pivot defines a longitudinal axis that is perpendicular to the longitudinal axis of the housing.

3. The surgical instrument of claim 1, wherein each of the first actuator and the second actuator of the first circuit is configured to control delivery of electrosurgical cutting energy.

4. The surgical instrument of claim 1, wherein the first pivot includes a longitudinally extending protrusion configured to fit within a corresponding recess defined by the housing.

5. The surgical instrument of claim 1, wherein the second pivot includes a longitudinally extending protrusion configured to fit within a corresponding recess defined by the housing.

6. The surgical instrument of claim 1, further comprising a suction and irrigation assembly operably coupled to the housing and including:
a dual chamber poppet valve defining a longitudinal axis extending at an angle relative to the longitudinal axis of the housing;
an irrigation button operably coupled to a first chamber of the dual chamber poppet valve and extending through an opening defined through a top surface of the housing; and
a suction button operably coupled to a second chamber of the dual chamber poppet valve and extending through an opening defined through a top surface of the housing.

7. The surgical instrument of claim 6, wherein the irrigation button and the suction button are disposed abreast on the top surface of the housing.

8. A surgical system comprising:
an electrosurgical generator configured to generate electrosurgical energy; and
a surgical instrument configured to operably couple to the electrosurgical generator, the surgical instrument including:
a housing;
a first assembly operably coupled to the housing and including:
a first function button pivotably coupled to the housing about a first pivot and including a first arm on a first side of the first pivot and a second arm on a second side of the first pivot; and
a first circuit supported by the housing and aligned with the first pivot between the first arm and the second arm, the first circuit including a first actuator on the first side of the first pivot and a second actuator on the second side of the first pivot; and
a second assembly operably coupled to the housing and including:
a second function button pivotably coupled to the housing about a second pivot; and
a second circuit supported by the housing beneath the second function button.

9. The system of claim 8, wherein the first pivot defines a longitudinal axis that is parallel to a longitudinal axis of the housing and the second pivot defines a longitudinal axis that is perpendicular to the longitudinal axis of the housing.

10. The system of claim 8, wherein each of the first actuator and the second actuator of the first circuit is configured to control delivery of electrosurgical cutting energy.

11. The system of claim 8, wherein the first pivot includes a longitudinally extending protrusion configured to fit within a corresponding recess defined by the housing.

12. The system of claim 8, wherein the second pivot includes a longitudinally extending protrusion configured to fit within a corresponding recess defined by the housing.

13. The system of claim 8, further comprising:
a suction system configured to generate vacuum pressure; and
an irrigation system storing irrigation fluid, wherein the surgical instrument is configured to couple to the suction system and the irrigation system, and the surgical instrument further includes a suction and irrigation assembly, the suction and irrigation assembly including:
a dual chamber poppet valve defining a longitudinal axis extending at an angle relative to the longitudinal axis of the housing;
an irrigation button operably coupled to a first chamber of the dual chamber poppet valve and extending through an opening defined through a top surface of the housing; and
a suction button operably coupled to a second chamber of the dual chamber poppet valve and extending through an opening defined through a top surface of the housing.

14. The system of claim 13, wherein the irrigation button and the suction button are disposed abreast on the top surface of the housing.

15. A surgical instrument comprising:
a housing;
a first assembly operably coupled to the housing and including:
a first function button pivotably coupled to the housing about a first pivot and including a first arm positioned on a first side of the housing and a second arm positioned on a second side of the housing; and
a first circuit including a first actuator a second actuator activatable by actuation of either of the first arm or the second arm of the first function button;
a second assembly operably coupled to the housing and including:
a second function button pivotably coupled to the housing about a second pivot; and
a second circuit supported by the housing beneath the second function button; and
a suction and irrigation assembly operably coupled to the housing and including:
a dual chamber poppet valve defining a longitudinal axis extending at an angle relative to the longitudinal axis of the housing;
an irrigation button operably coupled to a first chamber of the dual chamber poppet valve and extending through an opening defined on a top surface of the housing; and
a suction button operably coupled to a second chamber of the dual chamber poppet valve and extending through an opening defined on a top surface of the housing.

16. The surgical instrument of claim 15, wherein the first pivot defines a longitudinal axis that is parallel to a longitudinal axis of the housing and wherein the second pivot defines a longitudinal axis that is perpendicular to the longitudinal axis of the housing.

17. The surgical instrument of claim 15, wherein each of the first actuator and the second actuator of the first circuit is configured to control delivery of electrosurgical cutting energy.

18. The surgical instrument of claim 15, wherein the first pivot includes a longitudinally extending protrusion configured to fit within a corresponding recess defined by the housing.

19. The surgical instrument of claim 15, wherein the second pivot includes a longitudinally extending protrusion configured to fit within a corresponding recess defined by the housing.

20. The surgical instrument of claim 15, wherein the irrigation button and the suction button are disposed abreast on a surface of the housing.
